SEQUENCE LISTING (8 sequences)

SEQ ID NO: 1 - protein, 12 aa, human
  MOD_RES (1)..(1): pyroglutamic acid
EGPWLERPPPPC

SEQ ID NO: 2 - protein, 11 aa, human
  MOD_RES (1)..(1): pyroglutamic acid
EGPWLRPPPPC

SEQ ID NO: 3 - protein, 10 aa, human
  MOD_RES (1)..(1): pyroglutamic acid
EGPWRPPPPC

SEQ ID NO: 4 - protein, 12 aa, rat
  MOD_RES (1)..(1): pyroglutamic acid
ERPPLERPPPPC

SEQ ID NO: 5 - protein, 12 aa, human
  MOD_RES (1)..(1): pyroglutamic acid
ELGPQGRPPPPC

SEQ ID NO: 6 - protein, 15 aa, human
DPSKKQGPWLPPPPC

SEQ ID NO: 7 - protein, 9 aa, human
  MOD_RES (1)..(1): pyroglutamic acid
EGPWLEEEE

SEQ ID NO: 8 - protein, 7 aa, human or synthetic peptide
SSPPPPC

(19) United States
(12) Patent Application Publication
(10) Pub. No.: US 2006/0039911 A1
(43) Pub. Date: Feb. 23, 2006

(54) Method for the treatment of gastroesophageal reflux disease

(75) Inventors: Philip C. Gevas, Key Biscayne, FL (US); Stephen Grimes, Davis, CA (US); Stephen Karr, Davis, CA (US); Dov Michaeli, Larkspur, CA (US)

Correspondence Address: HOWSON AND HOWSON;ONE SPRING HOUSE CORPORATION CENTER, BOX 457, 321 NORRISTOWN ROAD, SPRING HOUSE, PA, 19477, US

(73) Assignee: Aphton Corporation, Philadelphia, PA (US)

(21) Appl. No.: 11/252,904

(22) Filed: Oct. 18, 2005

Related U.S. Application Data
(63) Continuation of application No. 10/314,057, filed on Dec. 6, 2002.
Application No. 10/314,057 is a continuation of application No. 09/700,378, filed on Mar. 1, 2001, now abandoned.
(60) Provisional application No. 60/085,610, filed on May 15, 1998.

(51) Int. Cl. A61K 39/395; A61K 38/22

(52) U.S. Cl. 424/145.1; 514/12

(57) ABSTRACT

A method for the treatment of gastroesophageal reflux disease comprising a combination of active immunization with an anti-gastrin immunogenic composition with an antagonist that blocks or inhibits gastric acid pump activity; or alternatively administering purified anti-gastrin antibodies with an H₂ antagonist or proton pump inhibitor of the gastric acid producing enzyme system.

### CROSS-REFERENCE TO RELATED APPLICATIONS

This application is a continuation of U.S. patent application Ser. No. 10/314,057, filed Dec. 6, 2002, which is a continuation of U.S. patent application Ser. No. 09/700,378, now abandoned, filed Mar. 1, 2001, which is a national stage of PCT/US99/10734, filed May 14, 1999, which claims benefit of the priority of U.S. Provisional Patent Application No. 60/085,610, filed May 15, 1998.

### BACKGROUND OF THE INVENTION

Gastroesophageal reflux disease (“GERD”) is a common and chronic disorder that requires long-term, even lifelong, therapy. GERD is commonly known as heartburn, which is characterized by a retrosternal burning sensation and regurgitation of the stomach contents. About 40% of adults in the United States have experienced occurrences of the disease, and approximately 10% have daily troubling symptoms.

GERD occurs when there is an abnormally prolonged contact time between the esophageal mucosa and refluxate, which is believed to be primarily gastric acid (DeVault, et al., Mayo Clinic Proc. 69.867-876, 1994 and Redmond, et al. In “Gastroesophageal Reflux Disease” Ronald Hinder ed., R. G. Landes Co., Ch. 1, pages 1-6, 1993). The regurgitation of the gastric contents and duodenal juice is believed to be due to either an incompetent lower esophageal sphincter or more frequently to an inappropriate sphincter relaxation at the time of transfer of the stomach contents between stomach and small intestine. The resultant reflux of acid and other materials from the stomach may induce pain or damage the esophageal mucosa. This damage to the esophageal mucosa may lead to esophagitis, which is characterized by inflammation of the esophageal mucosa, bleeding, cytological changes, peptic esophageal stricture, esophageal ulcer and Barrett's metaplasia, depending on the severity of the disease.

Gastric acid is produced by parietal cells in the stomach upon stimulation by acetylcholine, histamine and gastrin following the binding of each of these molecules with specific receptors on the surface of the cells. The peptide hormone gastrin is produced by mucosal cells in the stomach. Gastrin is secreted into the blood stream and is the most potent stimulant of acid secretion by the parietal cell. Gastrin is present in two molecular forms, heptadecagastrin (G17) and tetratriacontagastrin (G34). G17 is the primary stimulator of meal-induced gastric acid secretion and is 1500 times more potent than histamine, accounting for 60% of the gastrin-mediated acid release. It has also been found that in GERD patients having an abnormal sphincter, the postprandial levels of gastrin are twice those of a normal person and remain high, beyond 3 hours after the meal (Wetscher, et al. In Gastroesophageal Reflux Disease, R. A. Heinder, ed. R. G. Landes Co., Ch. 2, pages 7-29, 1993).

Normal esophageal pH is greater than pH 4. The acid refluxate from the stomach lowers the pH in the esophagus to less than 4, which results in damage to the esophageal mucosa and the development of GERD. In normal individuals, acidic refluxate is cleared by elimination of the refluxate by peristalsis of the esophagus and by neutralization of the acid with the bicarbonate produced by submucosal esophageal glands, and the bicarbonate present in swallowed saliva. In GERD patients, these mechanisms of acid neutralization are not sufficient to restore the normal esophageal pH values and prevent mucosal damage, since reflux of stomach contents occurs more frequently, and for a more prolonged period of time, than in normal individuals (Booth, et al., Arch. Surg. 96: 731-734, 1968 and Demeester, at al., Ann. Surg. 184: 459-470, 1976). Since it is not medically practical to alter the esophageal acid neutralization mechanisms, GERD therapies are directed to raising the pH of the stomach contents.

Currently, various therapies are available for the treatment of GERD. Historically, the medical treatment for GERD consisted of using antacids as acid neutralizing agents or antirefluxants, such as alginates, for alleviating an acute onset of the disease. However, these treatments are not effective for the therapy of chronic and severe symptoms of GERD. Systemic medications currently used for treating GERD include the histamine receptor antagonists, cimetidine and ranitidine, which are acid-suppressive agents directed to the inhibition of the four histamine type 2 (“H₂”) receptors. These agents prevent the normal binding of histamine, thereby inhibiting the parietal cell from secreting gastric acid and thus, they increase the pH of the stomach contents. The most commonly used histamine H₂ antagonists are cimetidine hydrochloride (TAGAMET™, SmithKline Beecham Pharmaceuticals), ranitidine hydrochloride (ZANTAC™, Glaxo Pharmaceuticals), fomatidine (PEPCID™, Merck & Co.) and nizatidine (Eli Lilly & Co.). The use of these H₂ antagonists is the standard treatment of acid-caused peptic disorders including GERD, since surgery, a more radical approach, is usually contraindicated.

Despite the widespread acceptance of histamine H₂ receptor blockers, controlled studies on GERD patients treated with these acid inhibiting compounds have yielded variable results on the healing of esophagitis and persistent symptomatic responses, such as continued acid production in the stomach. Studies using cimetidine and ranitidine in GERD patients, at doses and durations that had been proven effective in healing peptic ulcers, were not effective in GERD (Sabesin et al. Arch. Intern. Med 151:2394, 1991). At higher doses and duration of the H₂ antagonist therapy (400-800 mg and 150-300 mg twice daily, respectively, for cimetidine and ranitidine), approximately 50-70% (mean, 61%) of patients had symptomatic relief of GERD, and 0 to 82% (mean, 48%) had healing of their esophagitis as endoscopically determined (DeVault, et al. ibid, Koelz, H. R. Scand. J. Gastroenterol. 24:25-26, 1989 and Fennerty and Sampliner, Arch. Intern. Med. 151:2365-2366, 1991).

The healing of ulcerated or eroded esophageal mucosa requires a longer and more profound acid suppression than is necessary in treating other gastrointestinal ulcers. In patients in whom the symptoms of GERD disappeared after an effective treatment with histamine blockers, the symptoms of the disease reappeared soon after the treatment was discontinued (Antonson, et al. Gastroenterology 98: A16, 1990 and Bardhan, et al. Gastroenterology 98: A18, 1990).

Many patients with severe GERD hypersecrete gastric acid and may require high doses of H₂ antagonists, which become problematic in terms of patient compliance and long term use of these agents. The high doses of H₂ blockers when given to patients for a long period of time may cause undesirable side effects such as, blood pressure and heart problems. The increase in the effective dosage required to bring about relief of GERD symptoms results in very costly therapy. Although treatments of esophagitis vary widely depending on the severity of the disease, the more severe, high-grade types of the disease respond poorly to standard doses of histamine blockers. Approximately 50% or more of patients with GERD do not respond to histamine H₂ antagonist therapy and still require some other form of treatment. In addition, the effective treatment of GERD not only depends on increasing the concentration of histamine blockers or hydrogen pump inhibitors, which have also been found to be effective in the treatment of GERD, but effective dosing must be frequent, since the compounds have limited transient time in the patient and must be given in some situations approximately 4 times daily. In a significant number of cases, the patient is not responsive to H₂ blockers.

Proton pump inhibitors omeprazole (Astra AB), or anti-H⁺/K⁺-ATPase enzyme inhibitory compound, as well as its analogue, lansoprazole, (Takeda Chemicals) or pantoprazole (Byk Gulden) which inhibit acid secretion in the stomach by inhibiting the proton (hydronium ion) pump mechanism for producing hydrochloric acid in the parietal cell, have been found to be more effective than histamine H₂ blockers in alleviating the symptoms of GERD esophagitis. The resulting increase in pH induced by omeprazole leads to approximately 62-94% (mean, 83%) in symptomatic relief and a healing of the esophagitis occurs in 71-96% (mean, 78%) of the patients in 4-8 weeks of treatment for GERD, almost twice that of ranitidine (DeVault et al. ibid, Zeitoun, P. Scand. J Gastroenterology 166 (Suppl.): 83, 1989). A disadvantage of using omeprazole, lansoprazole, or pantoprazole similar to the case with histamine blockers, is that the compound must be administered at higher doses (20 mg twice daily or 40 mg once daily) than the dosages required to treat gastric and duodenal ulcers (20 mg once daily), and for a longer period of time in order to effectively treat GERD.

Furthermore, the prolonged use of histamine blockers or omeprazole for the treatment of GERD results in an increase in serum gastrin levels (2 to 4 times the basal level). It has been suggested that the increase in gastrin levels could lead to undesirable side effects such as dangerous trophic effects on the human gastric mucosa (Festen, et al. Gastroenterology 87: 1030-1034, 1984, Jansen, et al. Gastroenterology 99: 621-628, 1990 and Sontag, et al. Gastroenterology 102-109, 1992).

Co-assigned U.S. Pat. Nos. 5,023,077 and 5,609,870 disclose immunogenic compositions useful for controlling gastrin levels in a patient by generating anti-gastrin antibodies. Thus U.S. Pat. Nos. 5,023,077 and 5,609,870 disclose that the immunogenic compositions are useful for the treatment of gastric and duodenal ulcers and gastrin induced or responsive cancers and the disclosures are hereby incorporated entirely by reference into the present description.

There remains a need in the art for additional methods and compositions for the successful therapy of GERD.

### SUMMARY OF THE INVENTION

The present invention relates to a combination of immunological, antihormonal and enzyme inhibitory methods for the treatment of gastroesophageal reflux disease.

The invention combines a method for reducing gastric acid in the stomach by inhibiting the enzyme responsible for gastric acid production or secretion of gastric acid and an immunological method for reducing or preventing the increase of circulating gastrin. It is the object of the present invention to use anti-gastrin immunogenic compositions in the therapy of GERD in combination with administering effective doses of a proton pump inhibitor or H₂ antagonist so as to substantially raise the gastric pH while preventing elevated levels of circulating blood gastrin hormone.

This invention is directed to the treatment of GERD by gastric acid suppression by administration of a proton pump inhibitor or H₂ blocker together with the immunological reduction of circulating gastric hormone by neutralization of heptadecagastrin (G17) or tetratriacontagastrin (G34), or both G17 and G34 either by administration of exogenous specific antibodies or in situ by an immunogenic composition against gastrin.

It is the preferred embodiment of the invention to treat a patient with GERD by administering effective omeprazole dosages with effective dosages of antigastrin G17 antibodies.

It is the more preferred embodiment to keep the frequency of anti-gastrin immunogen parenteral administration to a patient suffering from GERD at a single effective dose or at least at only a few doses thereof.

Yet another preferred method of the invention is to pre-treat the GERD patient with gastrin immunogen or anti-G17 antibodies before administering the gastric acid producing enzyme inhibitor (i.e. proton pump inhibiting compound).

In one embodiment, the invention concerns a combination therapy with a histamine H₂ antagonist, such as ranitidine, cimetidine, fomatidine or nizatidine, or a proton pump inhibitor such as, omeprazole or lansoprazole, using standard dosing procedures for H₂ antagonist or proton pump inhibitor, respectively, as described by the art. In the preferred combination therapy, a patient is actively immunized with an immunological composition comprising gastrin 17(1-9)-h(G17)ser9-Diphtheria Toxin (see U.S. Pat. Nos. 5,023,077 and 5,468,494 (co-assigned). Once the patient is immunized, histamine H₂ antagonist or proton pump inhibitor therapy is administered for 2-12 weeks or until the desired serum anti-gastrin 17 antibody titer is reached. The novel combination therapy provides a more effective method for controlling acid output by the stomach, since acid production is thus controlled by two independent mechanisms, which results in a more effective method for treating GERD, including the more severe cases of the disease. In addition, the therapy would be a less costly method for treating GERD, without the problems with patient compliance associated with long term standard therapies. Furthermore, the high gastrin levels associated with standard therapies, particularly with omeprazole, are neutralized, and thus, the undesirable side effects are reduced.

The method of this invention for treating GERD permits a reduced dosage of the acid reducing agent both at the acid producing level as well as the acid production stimulating level (gastrin). This reduction of dosages is desirable in the usually prolonged treatment.

### BRIEF DESCRIPTION OF THE FIGURES

FIG. 1 illustrates experimental data concerning the percentage of time that the gastric contents remain above pH 3 in different groups of pigs treated with ranitidine (4 animals), omeprazole (5 animals) and hG17(1-9) (4 animals), as compared to six (6) control animals.

FIG. 2 illustrates the percentage of time that the gastric contents remain above pH 4 in a group of untreated (control) pigs (5) and groups of pigs treated with human gastrin 17(1-9)Ser9-Diphtheria Toxin (4 animals), ranitidine (4 animals) and omeprazole (5 animals) as described in FIG. 1.

FIG. 3 depicts the baseline median pH of the gastric contents of a group of six (6) untreated (control) pigs and groups of four (4) pigs treated with human G17(1-9)Ser9-Diphtheria Toxoid, three (3) pigs with ranitidine and five (5) pigs with omeprazole as described in FIG. 1.

### DETAILED DESCRIPTION OF THE INVENTION

The present invention relates to a novel combination of methods for the treatment of gastroesophageal reflux disease. The combined method on the one hand comprises inhibiting the normal binding of the hormone gastrin 17 to its physiological receptor by actively immunizing the patient against his or her own gastrin 17 hormone. Alternatively or additionally, the hormone gastrin 34 can be neutralized by active or passive immunization with G34 or C-terminal G17 peptide fragment. On the other hand, the method provides inhibition of production of gastric acid either by proton pump inhibition or H₂ receptor blockage.

The invention provides a novel immunological method for the treatment of gastroesophageal reflux disease using a peptide immunogen which raises sufficient gastrin 17 or gastrin 34 antibody levels in a patient so as to affect the binding of the gastrin 17 or gastrin 34 to its physiological receptors in the patient and raise the pH of the stomach. Gastric acid secretion in the stomach can thus be controlled. The pH of the stomach contents is simultaneously raised to a sufficient pH level, e.g., greater than pH 3 for a prolonged and sufficient period of time to alleviate the GERD symptoms and heal the acid-induced esophagitis. According to the invention, anti-G17 antibodies are induced in the patient by active immunization with peptide immunogens which comprise a G17 immunogen conjugated to an immunogenic carrier. The antibodies raised in the patient by the immunogens selectively and specifically bind gastrin hormone G17 or G34 or both, and neutralize and inhibit separately or together the normal binding of gastrin G17 or G34 or both to its receptors in the parietal cells, thereby controlling acid output in the stomach and preventing gastric acid damage of the esophageal mucosa during regurgitation.

A preferred embodiment of the inventive method provides a single administration of an active gastrin 17 or G34 immunogen, which has several advantages over the standard therapies of the art for treating GERD in that problems with patient compliance and undesirable side effects as a result of the therapy are eliminated. Other advantages of using the immunological methods for the treatment of GERD include the use of a limited number of administrations. A single primary administration with appropriately spaced boosters may last for approximately 6 months to a year. Another advantage is that, in a combination therapy with H₂ agonists or proton pump inhibitors, effective anti-gastrin 17 antibody titers can be maintained by occasional booster shots while the gastric acid inhibitor dosing is reduced or discontinued. Another advantage of this invention is that the maintenance of antigastrin antibody titers reduces or prevents excessive levels of gastrin in hypogastrinemia which would otherwise result from administration of a proton pump inhibitor or H₂ blocker. A booster shot of the immunological composition prolongs anti-gastrin 17 immunity and gastric acid suppression. Still another advantage of this method is that the immunization allows a sufficient time for the esophagitis to completely heal. Additionally, no surgery is required. Yet another advantage is that combination therapy is more useful for treating severe cases of GERD, without causing undesirable side effects, since excess serum gastrin 17 peptides are physiologically neutralized. In patients where the GERD condition is alleviated, discontinuation of the booster dose may result in resumption of normal gastrin levels.

According to the method of the invention, an immunogen is prepared using peptides or chemical structures that mimic the amino terminal end of gastrin 17 or of gastrin 34. The immunogens and immunogenic compositions of the invention are those described in U.S. Pat. Nos. 5,023,077, 5,469,494 and 5,609,870. The disclosures of these issued patents are hereby incorporated by reference in their entirety. U.S. Pat. Nos. 5,023,077, 5,469,494, and 5,609,870 disclose compositions containing anti-gastrin 17 immunogens as well as anti-gastrin 34 immunogens and methods of using these compositions for the treatment of gastric and duodenal ulcers and gastrin responsive cancers.

In the present invention, effective dosages ranging from 0.1 mg to 5 g of the immunogenic composition are administered for the treatment of GERD combined with 10-80 mg daily dose of omeprazole. An effective dosage of the immunogenic composition is capable of eliciting an immune response in a patient and inducing antibody titer against human gastrin 17 within 1-3 months after immunization.

Effective treatment of GERD according to this method results in maintenance of the pH of the stomach contents above pH 3 or 4, and for a more prolonged period of time than with H₂ antagonist therapy. Maintenance of the stomach pH above 3 or 4 is essential in the treatment of GERD, since refluxate material having a pH below 2.0 causes esophagitis by protein denaturation and cell damage, and pH values below 2.5 triggers painful episodes in a patient. When the pH is maintained above 2.5, pain perception is almost nonexistent (Smith, et al Gastroenterology 96: 683-689, 1989) and damage to the esophageal wall is minimized.

The immunogens and immunogenic compositions of the invention typically induce specific antibody responses after a single administration. However, it may take several weeks or months for antibody titers to rise to the desired levels effective for the treatment of GERD.

Combination therapy with a histamine H₂ antagonist, such as ranitidine, cimetidine, fomatidine and nazatidine, or a proton pump inhibitor, such as omeprazole or lansoprazole, is designed so that a GERD patient is immunized with an immunogenic composition of the invention, and administration of H₂ antagonist is provided on a daily basis, at least once a day for the first 2-12 weeks of treatment or until the desired serum level of anti-gastrin 17 antibodies is obtained.

Desired anti-gastrin 17 serum levels range from 10 to 300 pmole/ml. Once the desired serum levels of anti-gastrin 17 antibody titer are obtained, as measured by ELISA or RIA, the non-immunological gastric acid inhibiting drug portion of the combination therapy may be reduced or discontinued.

In the following Examples, the anti G17 immunogenic composition, 150 mg ranitidine and 60 mg omeprazole were administered to pigs and the resulting changes in the pH of the stomach contents before and after treatment were measured. Specifically, following the stomach pH measurements of the untreated control state of each pig, the stomach pH of the same pigs was measured after the animals were treated with either ranitidine, or composition of human gastrin 17(1-9)-h(G 17)ser9-Diphtheria Toxoid (Gastrimmune), or omeprazole administered individually and at different times in each of four animals (pigs).

### EXAMPLES

### Example 1

Gastrin neutralization was achieved by using the immunological composition Gastrimmune which is composed of the amino terminal domain of gastrin-17 linked, via an amino acid or peptide spacer to diphtheria toxoid which acts as the immunogenic carrier. The antibodies raised by virtue of the design of the immunogen, cross-reacted with both amidated and glycine-extended gastrin-17, two known proliferative forms of gastrin.

Serum antibody titers rose within 2 weeks of the initial immunization to levels with an antigen binding capacity of >10⁻⁹M. The presence of anti-gastrin antibodies within the serum of Gastrimmune-immunized mice was confirmed by using an ELISA. As expected, no bound gastrin levels were detected in animals immunized with control immunogen.

### Example 2

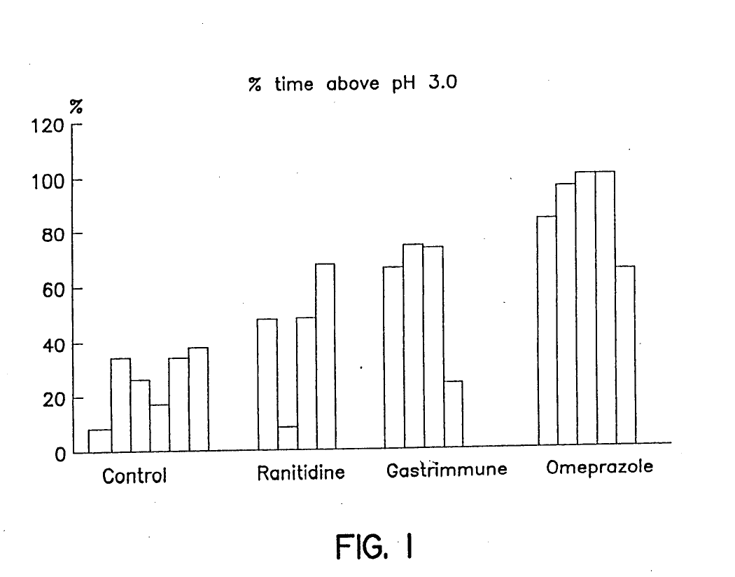

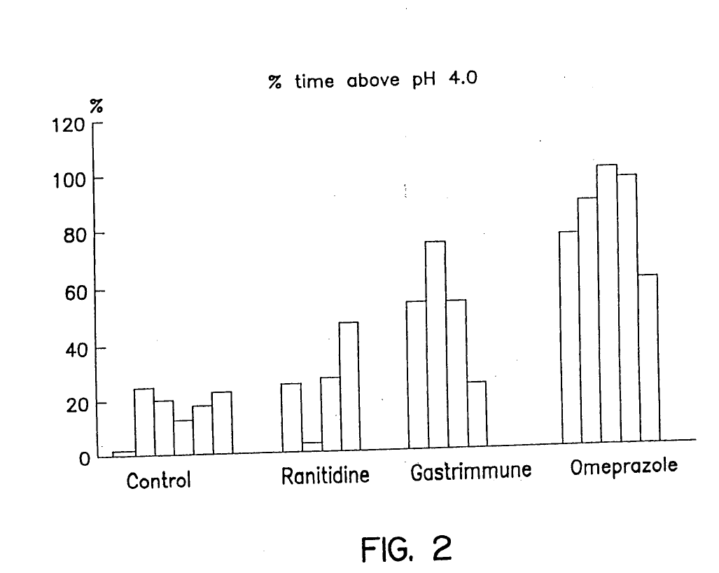

As can be seen in FIG. 1 and FIG. 2, the pH of the stomach contents remained above pH 3 or 4 in anti-gastrin 17 immunized pigs for a longer period of time than in the pigs treated with ranitidine. In omeprazole treated pigs the stomach pH was maintained above pH 3 or 4 for a longer period of time than pigs which were treated with ranitidine and anti-G17 immunized pigs.

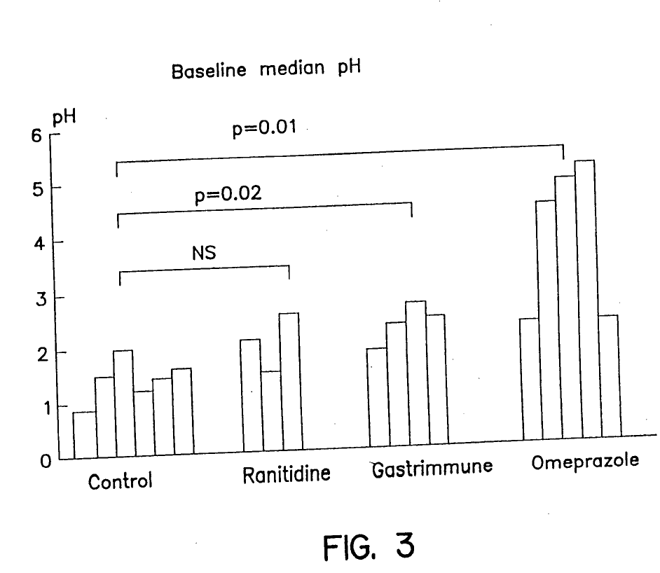

In addition, FIG. 3 shows the median pH exhibited by the stomach contents of control pigs when compared to ranitidine, anti-G17 immunization and omeprazole treatment. The data shows that the stomach pH is maintained at higher levels in pigs than those treated with ranitidine or anti-G17 immunization therapy. Anti-G17 immunized pigs had a median pH higher than ranitidine treated pigs.

Treatment of the pigs with ranitidine was less effective in preventing acid output from the stomach. Omeprazole treatment highly inhibited acid output. A single administration of anti-gastrin 17 immunization inhibited stomach acid output at a level of effectiveness between ranitidine and omeprazole, and sufficient to reduce the stomach acid output levels and increase the stomach pH for the effective treatment of GERD.

A treatment which combines the gastric acid secretion with proton pump inhibitors or H₂ histamine blockers with the novel immunization by e.g. Gastrimmune, can thus result in maintaining favorably raised pH in the stomach. Furthermore, the treatment with occasional, effective boosters of the antigastrin immunogenic composition can eventually, possibly within a few months, obviate any additional treatment with the anti-acid secretion drugs, such as e.g. omeprazole or ranitidine.

One of the possible advantages of the administration of a proton pump inhibitor or H₂ blocker after immunization with an antigastrin immunogen, as described, resides in the use of lower amounts of the proton pump inhibitor or H₂ blocker for effective lowering of gastrin acid secretion or raising of stomach pH to about 3-4.

### Example 3

The human patient suffering from GERD is immunized with 200-400 μg of primary i.v. inoculation of G17 (1-9) Ser DT immunogen composition. After 2 weeks a booster of 100-200 μg of the G17 (1-9) Ser DT composition is similarly administered. When the anti-G17 titer has reached a level of about 10-300 pmole/ml sufficient to lower the serum gastrin level to near normal with a concomitant lowering of gastric acid secretion, about 10-20 mg oral omeprazole preparation is administered daily to further reduce or stabilize the gastric secretion at a level which essentially eliminates or substantially ameliorates the GERD symptoms.

### Example 4

Immunogens capable of inducing specific immune responses to either G17 or to G34 were prepared by standard solid state synthesis methods. Each peptide was characterized as to amino acid content and purity.

Peptides with the following amino acid sequences were synthesized:

Peptide 1—Human G 17(1-6) (“hG 17(6)”): pGlu-Gly-Pro-Trp-Leu-Glu-Arg-Pro-Pro-Pro-Pro-Cys [SEQ ID NO: 1]

Peptide 2—Human G17(1-5) (“hG17(5)”): pGlu-Gly-Pro-Trp-Leu-Arg-Pro-Pro-Pro-Pro-Cys [SEQ ID NO: 2]

Peptide 3—Human G17(1-4) (“hG17(4)”): pGlu-Gly-Pro-Trp-Arg-Pro-Pro-Pro-Pro-Cys [SEQ ID NO: 3]

Peptide 4—Rat G17(1-6) (“rG17(6)”): pGlu-Arg-Pro-Pro-Leu-Glu-Arg-Pro-Pro-Pro-Pro-Cys [SEQ ID NO: 4]

Peptide 5—Human G34(1-6) (“hG34(6)”): pGlu-Leu-Gly-Pro-Gln-Gly-Arg-Pro-Pro-Pro-Pro-Cys [SEQ ID NO: 5]

Peptide 6—Human G34(13-22) (“hG34/G17 combination”): Asp-Pro-Ser-Lys-Lys-Gln-Gly-Pro-Trp-Leu-Pro-Pro-Pro-Pro-Cys [SEQ ID NO: 6]

Each of these peptides were conjugated to amino groups present on a carrier such as Diphtheria toxoid (“DT”) via the terminal peptide cysteine residue utilizing heterobifunctional linking agents containing a succinimidyl ester at one end and maleimide at the other end of the linking agent.

To accomplish the linkage between any of SEQ ID NOs. 1-6 and the carrier, the dry peptide was dissolved in 0.1 M Sodium Phosphate Buffer, pH 8.0, with a thirty molar excess of dithiothreitol (“DTT”). The solution was stirred under a water saturated nitrogen gas atmosphere for four hours. The peptide containing reduced cysteine was separated from the other components by chromatography over a G10 Sephadex column equilibrated with 0.2 M Acetic acid. The peptide was lyophilized and stored under vacuum until used. The carrier was activated by treatment with the heterobifunctional linking agent, e.g., Epsilon-maleimidocaproic acid N-hydroxysuccinimide ester, (“EMCS”), in proportions sufficient to achieve activation of approximately 25 free amino groups per 10⁵ MW of carrier. In the specific instance of diphtheria toxoid, this amounted to the addition of 6.18 mg of EMCS (purity 75%) to each 20 mg of diphtheria toxoid.

Activation of diphtheria toxoid was accomplished by dissolving each 20 mg aliquot of diphtheria toxoid in 1 ml of 0.2 M Sodium Phosphate Buffer, pH 6.45. Aliquots of 6.18 mg EMCS were dissolved into 0.2 ml of Dimethyl Formamide (“DMF”). Under darkened conditions, the EMCS was added dropwise in 50 μl amounts to the DT with stirring. After 2 hours of incubation in darkness, the mixture was chromatographed on a G50 Sephadex column equilibrated with 0.1 M Sodium Citrate buffer, pH 6.0, containing 0.1 mM EDTA.

Fractions containing the EMCS activated diphtheria toxoid were concentrated over a PM 10 ultrafiltration membrane under conditions of darkness. The protein content of the concentrate was determined by either the Lowry or Bradford methods. The EMCS content of the carrier was determined by incubation of the activated carrier with cysteine-HCl followed by reaction with 10 mM of Elman's Reagent 5,5′dithio-bis (2-nitrobenzoic acid) 10 mM. The optical density difference between a blank tube containing cysteine-HCl and the sample tube containing cysteine-HCl and carrier was translated into EMCS group content by using the molar extinction coefficient of 13.6×10³ for 5-thio-2-nitro benzoic acid at 412 nm.

The reduced cysteine content (—SH) of the peptide was also determined utilizing Elman's Reagent. Approximately 1 mg of peptide was dissolved in 1 ml of nitrogen gas saturated water and a 0.1 ml aliquot of this solution was reacted with Elman's Reagent. Utilizing the molar extinction coefficient of 5-thio-2-nitro-benzoic acid (13.6×10³), the free cysteine —SH was calculated. An amount of peptide containing sufficient free —SH to react with each of the 25 EMCs activated amino groups on the carrier was dissolved in 0.1 M Sodium Citrate Buffer, pH 6.0, containing 0.1 mM EDTA, and added dropwise to the EMCS activated carrier under darkened conditions. After all the peptide solution had been added to the carrier, the mixture was incubated overnight in the dark under a water saturated nitrogen gas atmosphere.

The conjugate of the peptide linked to the carrier via EMCS is separated from other components of the mixture by chromatography over a G50 Sephadex column equilibrated with 0.2 M Ammonium Bicarbonate. The conjugate eluted in the column void volume is lyophilized and stored desiccated at 20° C. until used.

The conjugate may be characterized as to peptide content by a number of methods known to those skilled in the art including weight gain, amino acid analysis, etc. Conjugates of these peptides and diphtheria toxoid produced by these methods were determined to have 20-25 moles of peptide per 10⁵ MW of carrier and all were considered suitable as immunogens for immunization of test animals.

### Example 5

Peptide hG17(1-9)-Ser9 was prepared by standard solid state synthesis methods. That peptide contains an amino terminal immunomimic of hG17 followed by a carboxy terminal spacer. This peptide comprises a 9 amino acid immunomimic of hG17 (pGlu-Gly-Pro-Trp-Leu-Glu-Glu-Glu-Glu-) [SEQ ID NO: 7] followed by the “Ser” spacer (-Ser-Ser-Pro-Pro-Pro-Pro-Cys-) [SEQ ID NO:8] attached to amino acid number 9 of the hG17 immunomimic.

The peptide was conjugated to amino groups present on the Diphtheria Toxoid (“DT”) immunogenic carrier via the terminal peptide cysteine residue utilizing heterobifunctional linking agents containing a succinimidyl ester at one end and maleimide at the other end of the linking agent essentially as described in Example 4.

The immunogenic constructs of this invention include an amino terminal (1-9) G17 peptide or an amino terminal (1-6) G34 peptide conjugated via a peptide spacer to an immunogenic carrier. The preferred G17 sequence is pyro-Glu-Gly-Pro-Trp-Leu-Glu-Glu-Glu-Glu [SEQ ID NO:7] and the preferred G34 sequence is pGlu-Leu-Gly-Pro-Gln-Gly-Arg-Pro-Pro-Pro-Pro-Cys [SEQ ID NO: 5]. The preferred spacer in both constructs is a Ser-peptide (Ser-Ser-Pro-Pro-Pro-Pro-Cys) [SEQ ID NO: 8]. The preferred immunogenic carrier is diphtheria toxoid, tetanus toxoid, keylimpet hemocyanin, and bovine serum albumin (BSA). The gastrin immunogen is defined as a conjugate of the pGlu-Gly-Pro-Trp-Leu-Glu-Glu-Glu-Glu [SEQ ID NO: 7] peptide sequence, with an amino acid spacer linked to an immunogenic carrier. The preferred gastrin immunogen is defined as a conjugate of the (1-9) amino terminal (pGlu-Gly-Pro-Trp-Leu-Glu-Glu-Glu-Glu) [SEQ ID NO: 7] peptide which is linked by peptide spacer to diphtheria toxoid.

Numerous modifications and variations of the present invention are included in the above identified specification and are expected to be obvious to one of skill in the art. Such modifications and alterations to the compositions and processes of the present invention are believed to be encompassed in the scope of the claims appended hereto.

1. A method for treating gastroesophageal reflux disease in a mammal comprising:
(a) administering to said mammal an immunogenic composition comprising
(i) a peptide comprising the amino terminal domain of gastrin-17 conjugated to an immunogenic carrier or
(ii) purified anti-gastrin antibodies that bind to gastrin, wherein said administration of (i) or (ii) provides anti-gastrin antibody levels in the serum of said mammal;
(b) administering periodically to said mammal an effective amount of an agent selected from the group consisting of a histamine H₂ antagonist and a proton pump inhibitor, and
(c) reducing or discontinuing the administration of (b) when said serum anti-gastrin antibody levels are within 10 to 300 pmole/ml.

2. The method according to claim 1, wherein said immunogenic composition comprises a pharmaceutically acceptable carrier.

3. The method according to claim 2, wherein said peptide of composition (i) is linked through an amino acid spacer to said immunogenic carrier.

4. The method according to claim 2, wherein said composition (i) induces anti-gastrin antibodies that bind to gastrin.

5. The method according to claim 1, wherein said antibodies bind to and neutralize heptadecagastrin (G17).

6. The method according to claim 1, wherein said antibodies bind to and neutralize tetratriacontagastrin (G34).

7. The method according to claim 1, wherein said antibodies comprise a mixture of antibodies that bind to and neutralize heptadecagastrin (G17) and antibodies that bind to and neutralize tetratriacontagastrin (G34).

8. The method according to claim 1, wherein said agent is a histamine H₂ antagonist.

9. The method according to claim 8, wherein said antagonist is selected from the group consisting of ranitidine hydrochloride, cimetidine hydrochloride, fomatidine, and nizatidine.

10. The method according to claim 1, wherein said agent is a proton pump inhibitor.

11. The method of according to claim 1, wherein said inhibitor is selected from the group consisting of omeprazole, lansoprazole and pantoprazole.

12. The method according to claim 1, wherein said agent is administered to said mammal until the serum anti-G17 antibody titer is 10-300 pmole/ml.

13. The method according to claim 1, wherein said immunogenic composition of step (a) is administered periodically to maintain said serum anti-gastrin antibody levels.

14. The method according to claim 1, wherein said immunogenic carrier is diphtheria toxoid.

15. The method according to claim 1, wherein said immunogenic composition comprises a peptide comprising the amino terminal domain of gastrin-17 conjugated to an immunogenic carrier.

16. The method according to claim 1, wherein said immunogenic composition comprises purified anti-gastrin antibodies that bind to gastrin.

17. A method for treating gastroesophageal reflux disease in a mammal comprising:
(a) administering to said mammal an immunogenic composition comprising a peptide comprising the amino terminal domain of gastrin-17 conjugated to an immunogenic carrier, wherein said administration induces anti-gastrin antibody levels in the serum of said mammal; and
(b) co-administering periodically to said mammal an effective amount of an agent selected from the group consisting of a histamine H₂ antagonist and a proton pump inhibitor.

18. A method for treating gastroesophageal reflux disease in a mammal comprising:
(a) administering to said mammal a composition comprising purified anti-gastrin antibodies that bind to gastrin; and
(b) co-administering periodically to said mammal an effective amount of an agent selected from the group consisting of a histamine H₂ antagonist and a proton pump inhibitor.
